(19) United States
(12) Patent Application Publication
(10) Pub. No.: US 2025/0377296 A1
(43) Pub. Date: Dec. 11, 2025

(54) Minimization of Noise in Optical Dat Capture for Liquids By Light Modulation Means

(71) Applicant: Airware, Inc., Newbury Park, CA (US)

(72) Inventors: Thomas G. Campbell, Newbury Park, CA (US); Jacob Y. Wong, Newbury Park, CA (US)

(73) Assignee: Airware Inc., Newbury Park, CA (US)

(21) Appl. No.: 19/312,087

(22) Filed: Aug. 27, 2025

Related U.S. Application Data
(63) Continuation of application No. 18/432,015, filed on Feb. 4, 2024, now abandoned.
Application No. 18/432,015 is a continuation-in-part of application No. 18/238,300, filed on Aug. 25, 2023, now Pat. No. 11,892,400.
Application No. 18/238,300 is a continuation-in-part of application No. 18/114,414, filed on Feb. 27, 2023, now Pat. No. 11,747,259.
Application No. 18/114,414 is a continuation-in-part of application No. 17/227,789, filed on Apr. 12, 2021, now Pat. No. 11,604,138.
Application No. 17/227,789 is a continuation of application No. 17/073,297, filed on Oct. 17, 2020, now Pat. No. 10,976,243.
Application No. 17/073,297 is a continuation-in-part of application No. 16/600,466, filed on Oct. 12, 2019, now Pat. No. 10,983,046.

(51) Int. Cl. G01N 21/3577; A61B 5/145; G02B 5/30

(52) U.S. Cl. CPC G01N 21/3577; A61B 5/14532; G02B 5/3025

(57) ABSTRACT

A process and sensor system with particular control of polarization of the interrogating light beams useful for determining a concentration of a targeted molecule M (such as glucose) within a given time period in a liquid sampling matrix through use of a Direct Infrared Laser Absorption Spectroscopy Technique.

### CROSS-REFERENCE TO RELATED APPLICATIONS

The present invention is a continuation of U.S. Ser. No. 18/432,015, filed Feb. 4, 2024, which is a continuation-in-part application of U.S. Ser. No. 18/238,300, filed Aug. 25, 2023, which is a continuation-in-part application of U.S. Ser. No. 18/114,414, filed Feb. 27, 2023, which is a continuation-in-part application of U.S. Ser. No. 17/227,789, filed Apr. 12, 2021, which is a continuation application of U.S. Ser. No. 17/073,297, filed Oct. 17, 2020, which is a continuation-in-part application of U.S. Ser. No. 16/600,466, filed Oct. 12, 2019, which is a continuation of U.S. Ser. No. 16/359,350, filed Mar. 20, 2019, which was a continuation-in-part application of U.S. Ser. No. 16/056,531, filed Aug. 7, 2018, which is a continuation-in-part of U.S. Ser. No. 15/785,829 filed Oct. 17, 2017, which is a continuation-in-part of U.S. Ser. No. 15/644,775 filed Jul. 8, 2017, which is a continuation in part of U.S. Ser. No. 15/594,418 filed May 12, 2017, which is a continuation-in-part application of U.S. Ser. No. 15/444,136 filed Feb. 27, 2017, which is a continuation-in-part application of U.S. Ser. No. 15/358,873, filed Nov. 22, 2016, the disclosures of all of which are specifically incorporated herein by reference.

### FIELD OF THE INVENTION

The present invention relates to various means to modulate light energy and control its delivery to heighten the precision of target analyte measurement in the liquid phase by absorption spectroscopy.

### BACKGROUND OF THE INVENTION

Non-Dispersive Infra-Red (NDIR) is a common and excellent measurement technique for detecting gases in the atmosphere. NDIR sensors utilize the principle that various gas molecules exhibit substantial absorption at specific wavelengths in the infrared radiation spectrum. The term “non-dispersive” as used herein refers to the apparatus used, typically a narrow-band optical or infrared transmission filter, instead of a dispersive element such as a prism or diffraction grating. The optical filter isolates the radiation in a particular wavelength band that coincides with a strong absorption band of a gas species for said gas species measurement.

The present invention builds upon past inventions disclosed in related applications to further advance the use of NDIR to detect molecules in a liquid medium.

This and further objects and advantages will be apparent to those skilled in the art in connection with the figures and the detailed description of the invention set forth below.

### SUMMARY OF THE INVENTION

The present invention is generally directed to systems and processes which employ a pulsed source signal which is circularly polarized to enhance determining a concentration of a targeted molecule M (such as glucose) within a given time period in a liquid sampling matrix by use of a Direct Infrared Laser Absorption Spectroscopy Technique. A pulsed reference signal and a pulsed interference signal may also be circularly polarized. The circular polarization can be configured to match a particular dichroism of a targeted molecule (such as the D-glucose enantiomer) or an interference target molecule or molecule populations in the sample.

In other aspects of the present invention, quantum dot lasers and polarization optics can be employed in producing polarized signals.

Accordingly, a primary object of the present invention is to provide an improved system and process for detection of molecules in a liquid medium.

This and further objects and advantages will be apparent to those skilled in the art in connection with the figures and the detailed description of the invention set forth below.

### BRIEF DESCRIPTION OF THE DRAWINGS

FIG. 1 illustrates the wave relationship between incoherent, coherent, coherent narrow band, and very coherent narrow band emission.

FIG. 2 illustrates light-emitting characteristics of three different light emitting technologies: LEDs with a broad wavelength emission band, Fabry-Perot lasers with a narrower wavelength emission band and Distributed Feedback lasers that can offer an extremely narrow wavelength emission band.

FIG. 3 illustrates an example of three wavelength emission bands that can be used to implement DILAST where there is a reference laser, a signal laser, and an interference laser.

FIG. 4 illustrates two chiral enantiomers of glucose.

FIGS. 5A and 5B illustrate how a horizontal polarizer and a vertical polarizer, respectively, affect unpolarized light.

FIG. 6 illustrates a quarter-wave plate designed to convert linearly polarized light into circularly polarized light.

FIG. 7 illustrates circularly polarized light imposed on the D-glucose chiral molecule with subsequent reduction in signal level by way of preferential absorption according to Beer's Law.

FIG. 8 illustrates how a circularly polarized element properly employed in a DILAST system will result in a greater level of optical absorption by the target analyte.

FIGS. 9A-C illustrate three different constructions for implementation of DILAST according to the present invention.

FIG. 10 illustrates a system concept of the present invention.

### DETAILED DESCRIPTION OF THE INVENTION

To aid the reader in reviewing the following detailed description of the invention, the following sets forth a table of contents with headings to identify where one is in the disclosure.

### Table of Contents

I. Description of DILAST (Direct Infrared Laser Absorption Spectroscopy Technique)
II. Laser Coherence & Polarization Benefits Use for Optical Interrogation
III. Current Art in Circular Polarization
IV. Application of Circular Polarization
V. Circular Dichroism
VI. System Design
VII. Application of feedback controls for phase matching when using multiple detectors with simultaneously pulsed beams
VIII. Optic Materials Control
IX. Addressing Simultaneous Pulsing and Interference

### I. Description of DILAST (Direct Infrared Laser Absorption Spectroscopy Technique)

Before addressing the merits of the present invention, it is worthwhile to review some of the disclosures the inventors have already made in DILAST, to gain a better understanding of the present invention.

U.S. Pat. No. 9,606,053 (2017) discloses an NDIR method which significantly suppresses scattering noise attributable to the much higher molecular density which is encountered in a liquid medium, as opposed to a gaseous medium. The method utilizes alternating and successively pulsing infrared radiation from signal and reference sources which are multiplexed and collimated into a single pulsed beam directed through the liquid sample. The pulse frequency is set sufficiently fast so as to provide almost the same molecular configuration to both the signal and the reference beams. The scattering noise encountered by both beams is effectively the same and can be significantly reduced through processing the ratio of their respective pass-through outputs.

U.S. Pat. No. 9,678,000 discloses using an NDIR method to detect glucose in a liquid medium. Glucose has an overtone absorption band located at 1,150 nm which can be used as the center wavelength for the signal beam. This absorption band is desirable because it has a water absorption coefficient of no greater than ˜1.0 cm⁻¹, which is especially preferred, as it helps to minimize effects created by water absorption. A reference beam wavelength of 1,064 nm, where there is no glucose molecule absorption, can be used as the center wavelength for the reference beam.

U.S. Pat. No. 9,726,601 discloses an improved NDIR method for determining the concentration of targeted molecules labeled M in a liquid medium admixed with interfering molecules labeled MJ which uses an additional interference radiation source besides those of the signal and reference to significantly reduce the interference noise. U.S. Pat. No. 9,823,185 (2017) discloses an improvement to this method with suppression of both scattering and absorption interference noise (AlN) via a reflection detection technique.

Fig. in U.S. Pat. No. 9,823,185 illustrates an optical setup of a Signal diode laser, a Reference diode laser and an Interference diode laser which are driven alternately and successively in groups of two by a 3-channel high speed waveform generator. The rest of the optical and electronic processing system setup for a three-diode laser system to suppress both scattering noise and AlN is the same as the two-diode laser system disclosed in U.S. Pat. No. 9,606,053 for suppressing just the scattering noise.

U.S. Pat. No. 10,475,586 discloses a signal source, an interference source, a reference source, a multiplexer and a collimator to pulse radiation in a pulsed beam which is detected by a detector as is described in greater detail in U.S. Pat. Nos. 9,606,053 and 9,823,185. The signal source emits radiation at a signal wavelength which is within a first absorption band of the targeted molecule M, the interference source emits radiation at an interference wavelength which is within a second absorption band of said at least one interfering molecule MJ, and the reference beam emits radiation at a reference wavelength which is neutral and is not within either the first absorption band or the second absorption band; at least one interfering molecule Mj absorbs radiation at the signal wavelength; and the signal source, the interference source and the reference source are each pulsed at a preselected frequency of at least N Hz which is sufficiently fast so that a given molecule of the targeted molecule M or said at least one interfering molecule MJ will not pass in and out of the liquid sampling matrix within the preselected frequency.

U.S. Pat. No. 10,041,881 discloses an improved NDIR method for liquids in which scattering noise is reduced and an Absorption Interference Noise (AlN) is suppressed with a reflection technique.

U.S. Pat. No. 10,241,044 discloses a process for deciding the validity of the calibration curve for targeted molecules MG in a liquid sample with interfering molecules. This value can further be used to adjust the calibration curve via a parameter linking the transmittances measured at the signal and interference wavelength channels in order to assure its validity.

U.S. Pat. No. 10,473,586 discloses different sample capture techniques to enhance the accuracy, precision and reliability of measurements with our inventive Direct Infrared Laser Absorptive Scattering Technique (DILAST) sensors.

U.S. Pat. No. 10,976,243 discloses configuring a sampling volume so that sampling error caused by changes of a targeted molecule passing in an out of the sample volume is approximately the same or less than a measurement error caused by an accuracy limit of the electronic components and the optical elements.

U.S. Pat. No. 11,604,138 discloses, among other things possible use of two detectors and obtaining a calibration curve when an interference beam is used to calculate the concentration level of a targeted particle, such as glucose, in a liquid sample.

U.S. Pat. No. 11,747,259 discloses increased precision for liquid absorption spectroscopy, especially for in vivo samples of human analytes, which is obtained by varying the signal or signal and interference central wavelengths when the temperature of the sample site varies beyond a selected threshold used for determining standardized signal or signal and interference central wavelengths.

U.S. Pat. No. 11,892,400 discloses passing a radiation beam into a liquid sample to a variable effective depth and, as values are determined for multiple effective depths, a sampling dataset is obtained which is used to calculate a concentration level of a targeted particle in the liquid sample by use a calibration dataset obtained from use of known samples.

Accordingly, for present purposes, it is worth noting that DILAST, in a basic form, provides a process for quantifying a concentration of a targeted molecule M in a liquid sample matrix through use of a sensor system comprised of electronic components, optical elements and software modules, comprising the steps of: (1) detecting, by at least one detector, a pulsed source signal, a pulsed interference signal, and a pulsed reference signal after they penetrate into a sample volume of the liquid sample matrix; (2) obtaining, using signal processing, i) a first average ratio value for a first preselected period of time from the pulsed source signal and the pulsed reference signal emerging from the sample volume and ii) a second average ratio value for a second preselected period of time from the pulsed interference signal and pulsed reference signal; and (3) calculating, using electronics, the concentration of the targeted molecule M in the liquid sample matrix based on the first average ratio value for the first preselected period of time and a calibration curve validated using the second average ratio value for the second preselected period of time, wherein a signal source emits radiation at a signal wavelength which is within a first absorption band of the targeted molecule M, an interference source emits radiation at an interference wavelength which is within a second absorption band of said at least one interfering molecule MJ, and a reference beam emits radiation at a reference wavelength which is neutral and is not within either the first absorption band or the second absorption band.

It is with this background that the present invention will now be discussed.

### II. Laser Coherence & Polarization Benefits Use for Optical Interrogation

Many light-emitting technologies are utilized for non-invasive investigation of materials spanning solids, liquids, and gases. With a focus on the investigation of liquids, it has been determined that the greater control that can be implemented with the light sources, so too will be the better result with the optical data gathered from the resulting captured light energy. Of the light-emitting sources available, lasers provide two important benefits other sources: 1) they typically emit coherent light energy and 2) that light energy is typically polarized. These properties are fundamental to the operation of lasers and are key factors in their usefulness across various applications.

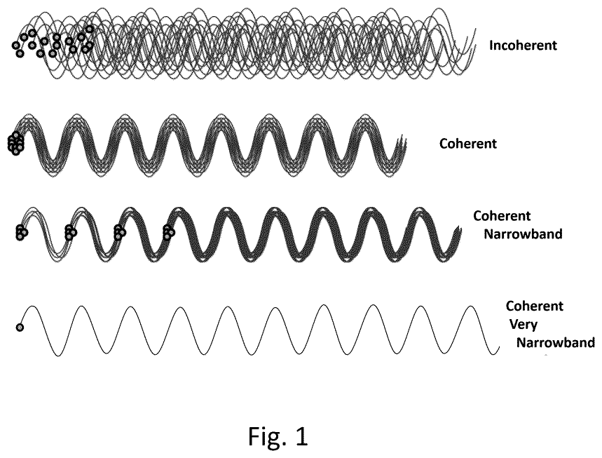

Coherence refers to the property of light waves emitted by a laser being in phase with each other, meaning they have a fixed phase relationship. This coherence allows laser light to exhibit interference phenomena and maintain its spatial and temporal properties. Coherent laser light is crucial for applications such as holography, interferometry, optical communication, and precision measurements. FIG. 1 diagrams the wave relationship between incoherent, coherent, coherent narrow band, and very coherent narrow band emission. It is clearly seen that with increasing levels of coherence of light energy, the more orderly is the output. And with optical interrogation of liquids, starting with the greatest level of energy control is advantageous.

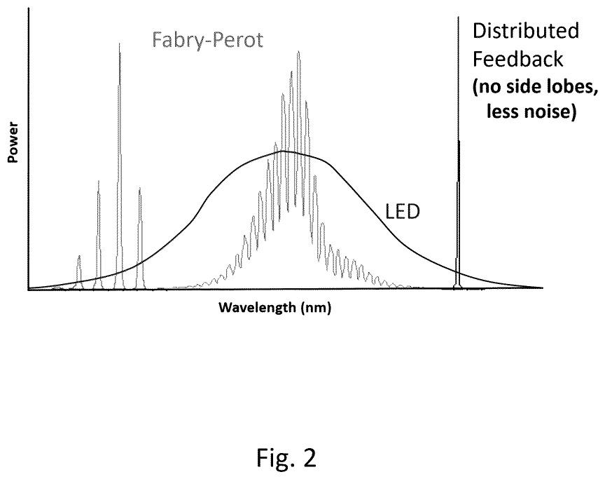

This energy control also manifests with the choice of light-emitting sources and the degree to which their primary emitted wavelengths can be controlled. In FIG. 2, representations are made of three light emitting technologies: 1) Light Emitting Diodes (LEDs) with broad wavelength emission, 2) Fabry-Perot lasers with narrower wavelength emission, and Distributed Feedback (DFB) lasers that can offer extremely narrow wavelength emission bands. For the purposes of analyte concentration measurement in liquids, it has been determined that a narrow band emitter tuned to specific absorption peaks or null absorption regions is beneficial for precision, accuracy, and repeatability of measurements.

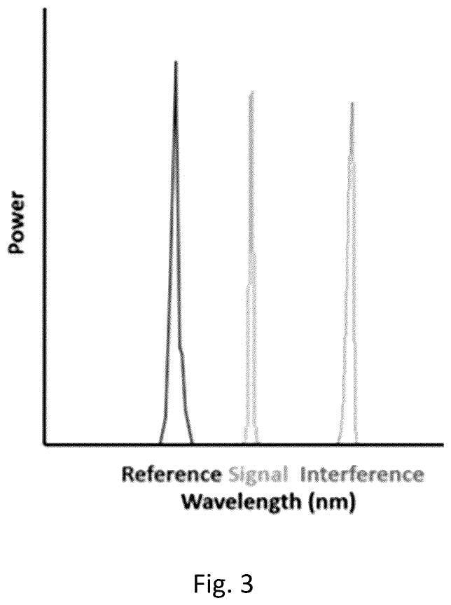

An example of the laser choices for implementing DILAST is shown in FIG. 3. Here representation is made of the relationship between a reference laser, a target analyte or signal laser, and an interfering compound or interference laser all of which exhibit very narrow band performance that can be controlled well below +/−5 nm of wavelength tolerance.

Now with a well-defined narrow-band energy source, we can describe the benefits for polarization of the energy from these and other emitting sources. Polarization refers to the orientation of the electric field vector of light waves. Most laser emitter light energy is polarized to some degree, meaning the electric field vectors of the light waves oscillate in a specific direction. The polarization can be linear, circular, or elliptical, depending on the orientation and phase relationship of the electric field vectors.

Polarization offers several benefits for the spectroscopy of liquids. 1) Enhanced Sensitivity: Polarization spectroscopy can enhance sensitivity to specific molecular orientations or structural features within the liquid sample. By selectively probing certain molecular transitions or electronic states, polarization-sensitive techniques can provide detailed information about molecular symmetry, alignment, and dynamics. 2) Selective Detection: Polarization-sensitive spectroscopy allows for selective detection of specific molecular transitions or electronic states that exhibit polarization-dependent behavior. This selective detection can help identify and characterize different molecular species or conformations within the liquid sample, improving the specificity and accuracy of spectroscopic measurements. 3) Structural Information: Polarization spectroscopy provides valuable structural information about the molecular arrangement, symmetry, and orientation within the liquid sample. By analyzing the polarization properties of the emitted or transmitted light, researchers can infer details about molecular geometry, intermolecular interactions, and molecular dynamics in solution. 4) Discrimination of Components: Polarization-sensitive spectroscopy supports discrimination between different components or species present in the liquid sample based on their polarization-dependent optical signatures. This capability is particularly useful for analyzing complex mixtures or multi-component systems, where individual components may exhibit distinct polarization properties. 5) Suppression of Background Signals: Polarization techniques can help suppress background signals and noise arising from non-specific interactions or scattering processes in the liquid sample. By selectively detecting polarization-dependent signals associated with specific molecular transitions, polarization spectroscopy can improve signal-to-noise ratios and enhance the detection sensitivity for target analytes. 6) Insight into Molecular Dynamics: Polarization spectroscopy can delve into the dynamic behavior and rotational motion of molecules within the liquid sample. Changes in polarization properties over time or under different experimental conditions can reveal information about molecular reorientation, rotational diffusion, and conformational dynamics in solution.

Both the coherence and polarization properties of laser light can be manipulated and controlled through various means, including laser cavity design, optical components, and external polarization control elements. By tailoring the coherence and polarization characteristics of laser light, we can tailor our laser systems for low-noise and high analyte specificity for precision concentration measurement in liquids.

When certain target analyte molecules exhibit chiral properties, this can be advantageously used for optical interrogation by matching a circular polarization to the chiral form of the target analyte for enhanced absorption and minimized unwanted reflections in a liquid system.

### III. Current Art in Circular Polarization

Current art with polarization techniques applied to analyzing biological tissues includes Optical Coherence Tomography (OCT), Polarization-Sensitive OCT (PS-OCT), and polarization speckle imaging. These are all coherence-based imaging modalities aimed at better characterizing human skin.

Coherent light and tissue birefringence are related to the phenomenon of polarization. Coherent light refers to light waves that have a fixed phase relationship with each other: a consistent frequency, amplitude, and phase. This coherence allows the light waves to exhibit interference phenomena and maintain their phase relationships over relatively long distances. Tissue birefringence, on the other hand, refers to the property of biological tissues to exhibit double refraction of light. Birefringence occurs when a material has different refractive indices for light polarized in different directions. When light passes through a birefringent material, it splits into two polarized components, each traveling at different speeds and refracting at different angles. This birefringence arises from the anisotropic structure of tissue components, such as collagen fibers, muscle fibers, nerve fibers, tendons, and cartilage.

When coherent light passes through a birefringent tissue, its polarization state can be affected by the tissue's optical properties. Depending on the orientation and arrangement of the tissue structures, the light waves may split into two orthogonally polarized components, each traveling at different speeds and refracting at different angles. This phenomenon is known as birefringence-induced polarization changes.

The relationship between coherent light and tissue birefringence enables techniques such as polarization-sensitive imaging and optical coherence tomography (OCT) to visualize and quantify tissue birefringence non-invasively. These techniques use coherent light sources and polarization-sensitive detectors to measure the changes in polarization induced by birefringent tissues. By analyzing the polarization properties of the light backscattered or transmitted through the tissue, researchers and clinicians can gain insights into tissue structure, organization, and health status.

The ability to quantify tissue birefringence can provide valuable information about tissue structure, organization, and health. For example, changes in tissue birefringence may occur due to pathological conditions, such as fibrosis, inflammation, or tissue remodeling. Understanding tissue birefringence can thus be important for biomedical research, clinical diagnosis, and monitoring of diseases and treatment outcomes.

Linear birefringence and optical activity speckle imaging are two coherence-based imaging modalities being applied to rapid non-invasive measurement of human tissue. The study of tissue birefringence can involve histology-like “thin-sample” transmission geometries and “thick-tissue” reflection geometries. Both modalities are sensitive to tissue morphology and have polarization-sensitive augmentations. For our application in studying tissue to determine target analyte concentrations, we do not employ OCT, PS-OCT, or Linear Birefringence, or Polarization Speckle measurement techniques. The approach described in this work refines the control and application of interrogating light energy to minimize overall optical noise in the sample such that we bring noise cancellation to higher levels with customized polarization of the light energy.

To better understand the relationship between polarization and coherence properties of skin, we must examine the way light interacts with tissue's structure and composition. Skin is complex with various structural components, including collagen fibers, elastin fibers, and cellular structures. These components exhibit birefringent properties. Collagen, for example, is a major structural protein in the skin and is highly birefringent due to its organized, fibrillar structure. As light interacts with collagen fibers and other tissue components, it undergoes changes in polarization, which can be quantified and analyzed to extract information about the tissue's structure, organization, and health.

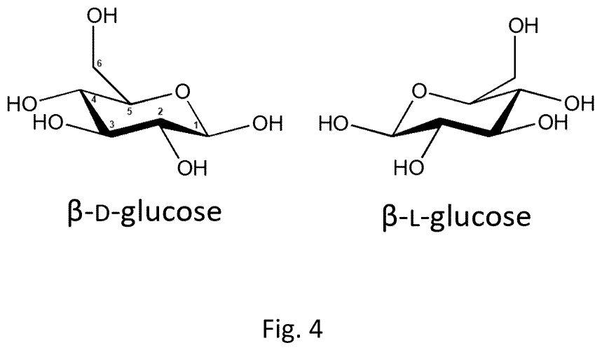

Within this environment, coherent light, with its fixed phase relationship between different parts of the wavefront, brings a certain level of noise control within the sample. For example, coherence enables high-resolution imaging techniques like optical coherence tomography (OCT) to visualize tissue microstructure and detect subtle changes associated with disease or injury.

Whereas other techniques leverage the polarization sensitivity of skin tissue to extract typically image-based information, this invention utilizes polarization to enhance the cancellation of common Mie, Raleigh, and geometric scattering noise by matching the type of polarization between the reference light beam and the target light beam as previously disclosed. For example, with enhanced polarization control, the resultant reflections of light energy from the D-glucose enantiomer, which is the primary chiral form in the human body presented in FIG. 4, will be more uniform and thus likely to be canceled with the previously disclosed ratio method. The resultant detected delta value between the reference light beam and the target analyte light beam delivers a higher level of precision in the target analyte concentration determination with absorption spectroscopy.

Multiple scattering of light in biological tissues rapidly scrambles polarization, so polarimetric biomedicine is challenging. There are ways to mitigate these and other tissue-specific challenges to extract useful biophysics via both Stokes and Mueller matrix analyses for “thin-sample” transmission geometry and “thick-tissue” reflection geometry as is discussed in Alex Vitkin, Photon mayhem: polarized light for structural and functional assessment of biological tissues Proceedings Volume PC12690, Polarization Science and Remote Sensing XI; PC1269003 (2023) https://doi.org/10.1117/12.2683831, the disclosure of which is specifically incorporated herein by reference.

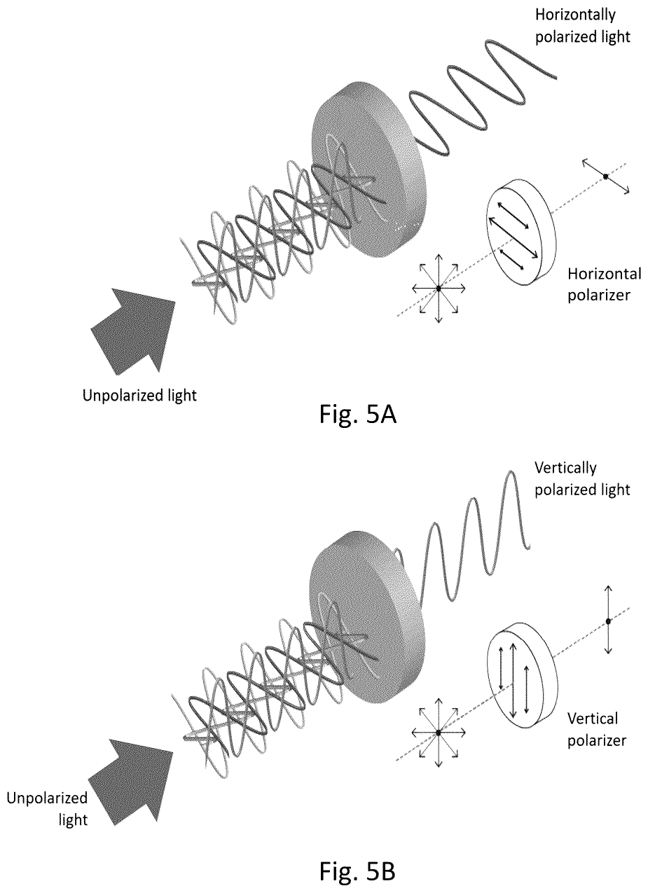

Adding a polarizer to a laser beam can have various effects on the beam output, depending on the specific characteristics of the laser and the polarizer, as well as the application. FIGS. 5A and 5B indicate the function of a horizontal and a vertical polarizer, respectively, when placed in line with a non-polarized light beam. In fact, there are several details to consider: 1) Intensity Reduction: Polarizers typically absorb a portion of the incident light, leading to a reduction in the intensity of the laser beam. To implement DILAST, dissipation of high power is typically not a problem as power levels are sub-Watt. 2) Beam Distortion: Improper alignment or low-quality polarizers may introduce beam distortion, scattering, or aberrations, affecting the spatial and temporal coherence of the laser beam. This can lead to a reduced signal-to-noise ratio. 3) Depolarization: Some polarizers may not provide perfect polarization extinction, leading to residual unpolarized or partially polarized light in the beam. This effect can impact the accuracy and reliability of polarization-sensitive measurements or experiments. 4) Compatibility Issues: Care must be taken to match the nature of the polarizer to the laser system. 5) Alignment Challenges: Achieving precise alignment between the laser beam and the polarizer axis is essential to maximize polarization efficiency and minimize unwanted effects. 6) Enhancement of Polarization: If the laser light is not perfectly polarized or if the desired polarization state differs from the laser output, aligning the polarizer to the desired polarization axis can enhance the polarization purity of the beam. 6) Polarization Control: By rotating the polarizer, the polarization axis and intensity of the beam can be adjusted. 7) Intensity Reduction: Depending on the orientation and properties of the polarizer, adding a polarizer to the laser beam may reduce the intensity of the beam. 8) Beam Quality: The addition of a polarizer may affect the beam quality and coherence properties of the laser light, possibly degrading the spatial and temporal coherence of the beam. Care must be taken to select and precisely align system components to minimize these effects.

### IV. Application of Circular Polarization

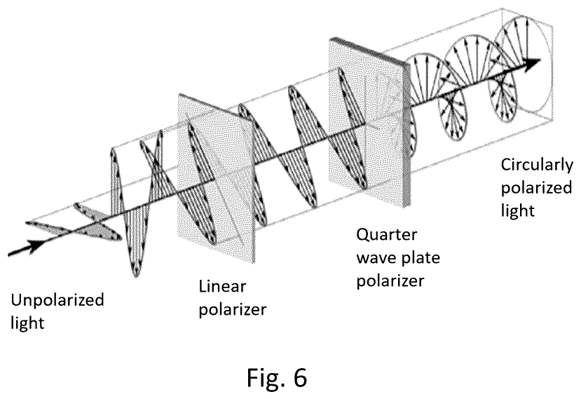

A circular polarizer manipulates the polarization state of light based on the principles of polarization and wave optics. Whereas linear polarization eliminates light or electromagnetic waves propagating in all directions except for the polarized plane of the polarizer utilized, a circular polarizer typically consists of a linear polarizer layer followed by a quarter-wave plate. The linear polarizer serves as the first component and polarizes the light by allowing only vibrations in a specific direction to pass through while blocking vibrations in other directions. As shown in FIG. 6, a quarter-wave plate is designed to convert linearly polarized light into circularly polarized light. A quarter-wave plate delays one polarization component of the incident light by a quarter of a wavelength with respect to the other component. Circularly polarized light consists of two perpendicular polarization components that are out of phase with each other, creating a helical wavefront.

Of light energy useful for implementing DILAST precision, a laser beam is preferred as it is typically polarized and may only require the addition of a quarter wave plate in the system. The degree and orientation of polarization depend on the specifics of the laser source. Quantum dot laser technology delivers wavelengths useful for the optical interrogation of human skin. These wavelengths are in regions of relatively low water absorption. Quantum dot lasers deliver desirable wavelengths, line widths, operating frequencies, precision pulsing, and power levels for DILAST applications. When a laser beam with polarized light passes through a circular polarizer, the effect depends on the alignment of the polarizer's polarization axis with respect to the polarization direction of the incoming light. One downside of the use of a circular polarizer is the attenuation of the light energy.

The polarization of a laser beam arises from various factors, including the design of the laser cavity, the properties of the laser medium, and any polarization optics used within the laser system. In many cases, the polarization of a laser beam is linear, meaning the electric field oscillates in a specific direction along the beam axis. However, the exact polarization characteristics of a laser beam can vary depending on the type of laser, its construction, and any optical elements it passes through. Some lasers naturally emit highly polarized light, while others may emit light with varying degrees of polarization or even unpolarized light.

Quantum dot lasers can exhibit coherence in their light output, but whether they are naturally coherent depends on various factors related to their design, operation, and the specific characteristics of the quantum dots used. Coherence in lasers refers to the property of emitting light waves that maintain a fixed phase relationship over time and space. Quantum dots are semiconductor nanocrystals with unique optical and electronic properties. The size, shape, and composition of quantum dots can affect their emission characteristics, including coherence properties. Coherence in lasers is also closely related to the gain spectrum of the laser medium, which determines the frequencies of light that the laser can emit. Quantum dot lasers can have broad gain spectra, which may limit their coherence properties compared to traditional semiconductor lasers with narrower gain spectra.

Approaches are being explored to improve the coherence properties of quantum dot lasers include internal and external cavity design, temperature control, injection current control, stabilization techniques, and hybrid integration. Optimizing the design of the laser cavity can help control the spectral properties and coherence characteristics of quantum dot lasers. This includes selection of cavity length, mirror reflectivity, and mode confinement to enhance coherence and minimize linewidth broadening effects. Temperature strongly influences the gain spectrum and coherence properties of quantum dot lasers. Maintaining stable temperature conditions and implementing temperature tuning techniques can help optimize the spectral characteristics and coherence of the laser output. Precise control of the injection current into the quantum dot laser can influence its gain spectrum and coherence properties. Modulating the injection current profile and using current stabilization techniques can help improve coherence and spectral purity. Implementing active stabilization techniques, such as optical feedback control or self-injection locking, can help enhance the coherence and linewidth narrowing of quantum dot lasers. These techniques provide feedback mechanisms to stabilize the laser output and suppress linewidth broadening effects. Employing external cavity configurations, such as distributed feedback (DFB) or distributed Bragg reflector (DBR) structures, can enable precise control over the laser emission spectrum and enhance coherence properties. Lastly, integrating quantum dot lasers with other optical components or materials, such as photonic crystals or resonant cavities, can help tailor the gain spectrum and coherence characteristics for specific applications. All refinements benefit from advances in quantum dot material engineering, such as size and composition control, surface passivation, and defect engineering, which can improve the spectral purity and coherence properties of quantum dot lasers.

Noted here, multiple scattering of light in biological tissues rapidly scrambles polarization, so polarimetric biomedicine and use for industrial chemical production and quality control is challenging. Optical interrogation methods intent on image creation rely upon data extraction via both Stokes and Mueller matrix analyses. For DILAST absorption spectroscopy concentration determinations, analyses typically do not rely on matrix calculations, however, this may prove useful should multiple light sources with various wavelengths be employed in the future for simultaneous interrogation of the sample.

### V. Circular Dichroism

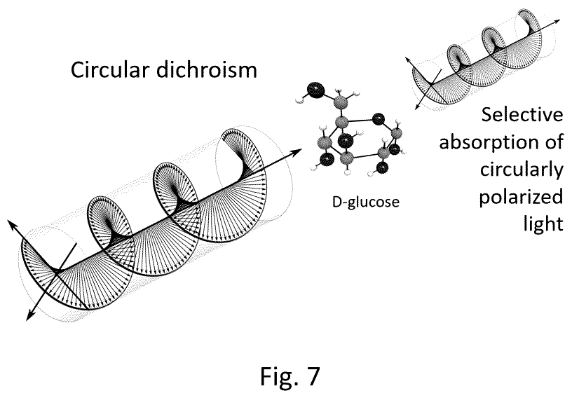

Circular dichroism (CD) is a spectroscopic technique used to study the chiral properties of molecules, particularly those found in organic and biological compounds. It measures the difference in the absorption of left-handed circularly polarized light (L-CPL) and right-handed circularly polarized light (R-CPL) by a sample as a function of wavelength. The principle lies in the differential absorption of circularly polarized light due to the chiral nature of molecules. Chiral molecules, which lack internal planes of symmetry, interact differently with left-handed and right-handed circularly polarized light because of their handedness. Revealed in FIG. 7 is circularly polarized light imposed on the D-glucose chiral molecule with subsequent reduction in signal level by way of preferential absorption according to Beer's Law.

In CD spectroscopy, chiral molecules exhibit electronic transitions influenced by their three-dimensional structure. As the molecules absorb circularly polarized light, these electronic transitions lead to characteristic absorption bands in the CD spectrum. CD spectroscopy is highly sensitive to the chirality of molecules and can provide information about their structural and conformational properties. It is particularly useful for analyzing biomolecules such as proteins, nucleic acids, and carbohydrates, which often have complex chiral structures and exhibit specific CD signatures associated with their secondary and tertiary structures.

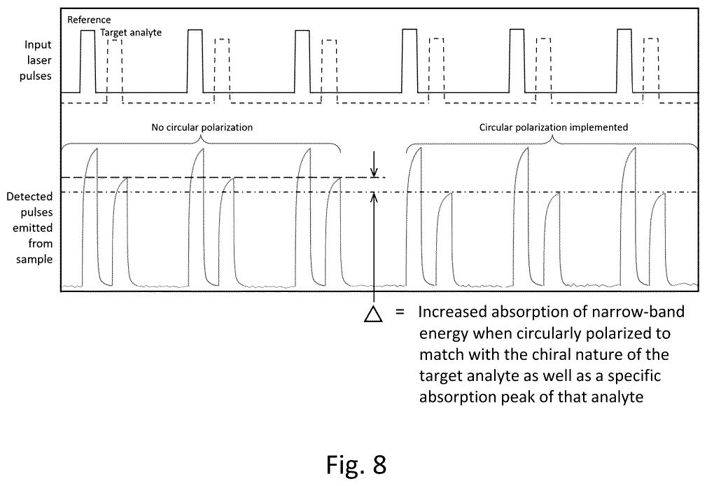

The CD spectra can be quantitatively analyzed to extract structural information, such as the percentage of secondary structure elements present in a protein sample or the degree of helical folding in nucleic acids. In this invention, the ability to enhance the degree of target analyte absorption while minimizing deleterious reflection activity is presented. As represented in FIG. 8, when a circularly polarized element is properly employed in the DILAST system, a greater level of optical absorption by the target analyte will result.

### VI. System Design

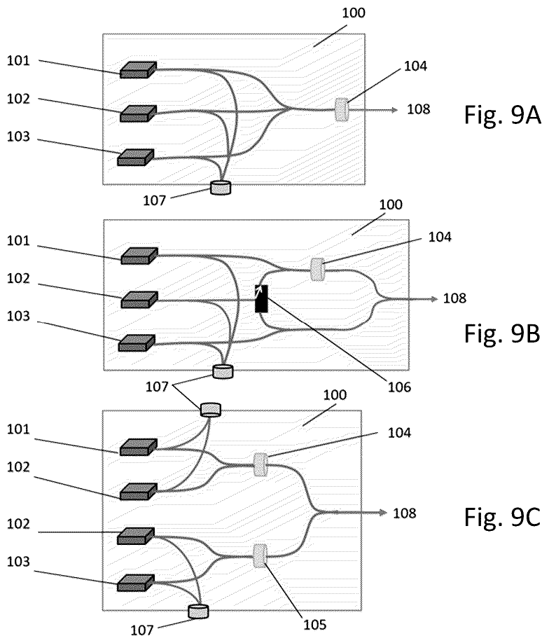

Several different constructions for implementation of DILAST according to the present invention are illustrated in FIG. 9A-C. several constructions are presented in FIGS. 9A-C.

For this level of device integration, that does not limit the further wafer level integration, packaged laser devices are mounted onto planar waveguides. The planar waveguides provide a carrier function for follow-on optical components as well as various optical combining and/or coupling channels with the result being a single output beam delivering pulsed light energy to the sample under interrogation.

In FIG. 9A, planar waveguide (100) supports three laser devices, the target analyte laser (101), the reference laser (102) and the interference laser (103). Outputs of all three lasers are combined and directed through a circular polarizer (104) prior to exit from the planar waveguide. The lasers are individually monitored by one or more photodetectors (107).

In FIG. 9B, consideration is made where the circular polarization is not used with the reference and interference lasers. Here optical switch (106) directs the pulsed output of the reference laser (102) either to pulse with the target laser (101), both passing their energy through the circular polarizer (104), or directs the reference laser (102) to pulse with the interference laser (103) with no circular polarization applied to their output.

In FIG. 9C, consideration is made for a separate circular polarizer (105) to be implemented for particular circular dichroism matching of the reference (102) and interference (103) lasers to the interference target molecule or molecule populations in the sample.

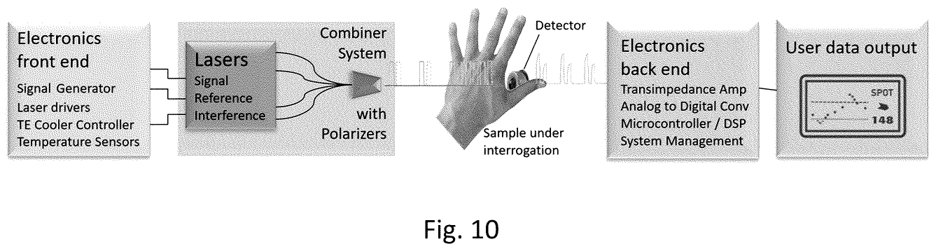

In FIG. 10 we see a system concept wherein the lasers and polarizers are integrated tightly with the optical combining function provided by the planar waveguide to deliver precise optical pulses to the sample for follow-on detection and signal processing.

By implementing a planar waveguide over discrete components connected by fiber optics, one can secure many benefits. First off is dramatic size miniaturization whether the components are mounted onto the planar waveguide or integrated within the planar waveguide structure. Second, lower optical losses are ensured through fewer discrete interconnections and pure consistency presented by precise optical fabrication processes used to build planar waveguides. Third, and although our optical pulsing timescales are quite slow as compared to the telecommunications world, the optics paths presented by planar waveguides provide tight confinement of light within the waveguide structure, reducing dispersion effects and supporting high-bandwidth transmission if necessary. Fourth, planar waveguides offer reduced crosstalk between adjacent optical components compared to discrete fiber optic components improving signal isolation. Fifth, by permitting relatively close co-location of the driver circuitry, lasers, and optic elements, the challenge of precise temperature control is simplified with the use of a single thermoelectric cooling (TEC) system or with distributed TECs space as necessary. The temperature stability with planar waveguides can be further enhanced by the choice of fabricating materials that have good heat transfer and, ideally match with the thermal coefficients of the components involved to minimize any structural strain for long term performance.

All of these factors weigh in on keeping the system costs to a minimum for both the fabrication side as well as the operational side of using such optical sensor systems, especially when battery operation and long life are market demands.

The choice of materials for constructing an optical planar combiner depends on factors such as wavelength range, optical properties, fabrication techniques, and desired performance characteristics. Some common materials used for optical planar combiners include: 1) Silicon (Si) which is widely used in photonic integrated circuits (PICs) and optical waveguides due to its compatibility with complementary metal-oxide-semiconductor (CMOS) fabrication processes. Silicon-on-insulator (SOI) substrates offer low propagation losses and high integration density, making them suitable for constructing planar combiners for applications in the near-infrared (NIR) and mid-infrared (MIR) spectral ranges. 2) Silica (SiO2) based materials, such as fused silica and silica-on-silicon (SOS) substrates, are commonly used in optical waveguides and integrated photonics platforms offering low optical losses, high transparency over a wide range of wavelengths, and compatibility with standard microfabrication techniques. 3) Glass substrates such as borosilicate glass and BK7 glass, are for planar waveguides and integrated optics, offering excellent optical transparency, thermal stability, and compatibility with photolithography and wet etching processes for a wide range of optical wavelengths. 4) III-V compound semiconductors such as indium phosphide (InP) and gallium arsenide (GaAs), used in active photonic & optoelectronic integrated circuits (OEICs) can deliver high optical gain, very high-speed, and compatibility with epitaxial growth techniques. 5) Glass-ceramics and photonic crystals offer tailored optical properties, including bandgap engineering, dispersion control, and nonlinear optical effects. 6) Polymer-based materials such as poly(methyl methacrylate) (PMMA) and polyimides offer low cost, flexibility, and ease of processing, making them suitable for constructing planar combiners for applications requiring lightweight and flexible optical components that may someday be needed in extreme physical forms such as a wearable ring or earring sensor.

VII. Application of Feedback Controls for Phase Matching when Using Multiple Detectors with Simultaneously Pulsed Beams

As revealed, the control and delivery of precise energy pulses to the sample and then the detection of the resultant outputs relies on thorough understanding of how the light energy is created and then subsequently manipulated for delivery to the sample. Phase control circuitry can exert significant control over the phase output of a quantum dot narrow-band laser, but the extent of control depends on various factors including the design of the laser system, the characteristics of the quantum dots, and the specific phase control techniques employed. Quantum dot lasers are semiconductor lasers where the active region consists of quantum dots, which are nanoscale semiconductor particles.

Phase control circuitry in quantum dot lasers typically operates by adjusting the electrical or optical parameters of the laser to control the phase of the emitted light. This phase control can be achieved through several methods: 1) Injection Current Control: By modulating the injection current into the laser diode, the carrier density and gain within the active region can be adjusted, which can influence the phase characteristics of the emitted light. 2) Temperature Control: Temperature changes can affect the refractive index and the gain characteristics of the laser cavity, thereby influencing the phase of the output light. Temperature control circuitry can adjust the laser temperature to control the phase behavior. 3) Feedback Control Systems: Feedback control systems, such as phase-locked loops (PLLs) or digital signal processing (DSP) algorithms, can monitor the phase of the laser output and adjust control parameters in real-time to maintain desired phase characteristics. 4) External Phase Modulation: External phase modulators can be integrated into the laser system to directly modulate the phase of the output light. This allows precise control over the phase characteristics and enables applications such as coherent communication systems.

The extent to which phase control circuitry can control the phase output of a quantum dot narrow-band laser depends on the sophistication of the control algorithms, the speed of the control mechanisms, and the stability of the laser system. In modern laser systems, phase control can be highly precise, allowing for applications that require precise phase control such as coherent communication systems, optical sensing, and metrology.

### VIII. Optic Materials Control

In concert with comprehensive control of the light emitters, key characteristics of the other optical elements must be controlled as well. Circular polarizers for near-infrared (NIR) wavelengths need to be designed with materials that have good optical properties within the NIR spectrum. Additionally, they should possess the ability to manipulate the polarization state of light effectively. Suitable materials for circular polarizers in the NIR range include: 1) Polymeric Films: Certain types of polymer films, such as polyvinyl alcohol (PVA) and polycarbonate, can be used to create circular polarizers for NIR wavelengths. These films can be stretched or oriented to induce birefringence, allowing them to polarize light effectively in the NIR spectrum. 2) Liquid Crystals: Liquid crystal materials can also be used to create circular polarizers for NIR wavelengths. Liquid crystal devices offer tunable polarization properties and can be designed to operate within specific NIR ranges. 3) Metal Nanoparticles: Metallic nanoparticles, such as gold or silver nanoparticles, can exhibit plasmonic resonance effects in the NIR spectrum. These nanoparticles can be incorporated into thin films or coatings to create circular polarizers that operate efficiently at NIR wavelengths. 4) Dielectric Metasurfaces: Dielectric metasurfaces composed of subwavelength nanostructures can manipulate the polarization of light across a broad range of wavelengths, including the NIR spectrum. These metasurfaces can be engineered to achieve circular polarization for NIR light with high efficiency. 5) Semiconductor Quantum Dots: Semiconductor quantum dots, such as cadmium selenide (CdSe) or lead sulfide (PbS) quantum dots, can be used to create circular polarizers for NIR wavelengths. Quantum dots offer tunable optical properties and can be synthesized to operate within specific NIR ranges.

Much of the art with uniquely fabricated metamaterial polarizers aims at relatively thin films of compounds being analyzed such as on slide samples put under microscopes. The application of a circularly polarized filter in conjunction with DILAST (Direct Infrared Laser Absorption Spectroscopy Technique) works to minimize the energy directed into considerably thicker, and typically more complex component-wise subjects under optical interrogation. The overall goal with the addition of the circular polarizer is then to minimize the optical noise naturally created in a liquid by the reflection effects from Rayleigh, Mie, and geometric scattering. We match a tailored, circularly polarized pair of reference and target analyte energy beams traveling through or reflected out of a liquid sample containing a particular chiral target analyte form. The commonality of the chirality of reference beam to the target analyte beam makes the resulting Rayleigh, Mie and geometric scattering more common enhancing our overall noise cancellation, keeping in mind that our extremely narrow band approach minimizes effects of optical absorption by the bulk of the constituents in the complex sample. An added benefit is enhancing the energy absorption of the polarized target analyte beam matched to the chiral target analyte.

In addition to detecting the transmitted or reflected energy from the sample under interrogation by means of DILAST with applied circular polarization on the reference and target analyte energy beams, we can alternately apply that circular polarizer for an extended data set that may reveal enhanced information on the target analyte concentration. Also, temporary removal of the circularly polarized filter may prove beneficial when implementing the periodic “interfering compound” pulse paring described in our U.S. Pat. No. 9,726,601. This method of tracking interfering compounds relies upon a common absorption peak for both protein and lipids as a group at 1210 nm. With this grouping of molecules, there is much less consistency, if any, in regard to a dominant chiral molecular structure, so applying the circular polarization to the interfering interrogation energy beam may prove, with certain liquid samples, to create more reflective noise and be undesirable for achieving the highest signal-to-noise ratio possible.

### IX. Addressing Simultaneous Pulsing and Interference

Simultaneous or near-simultaneous pulsing of respective pulse pairs (signal source & reference beam and interference source & reference beam) provides several benefits with two or more detectors that are incorporated by way of 1) a beam splitter that directs wavelengths, for example from 1100 nm and lower to one detector and above 1100 nm to the other detector, 2) stacked detectors where the upper detector is transparent to the wavelengths detected by the lower detector, and 3) adjacent detectors each receiving part of the same captured energy such that one detector registers the reference beam wavelength of 1064 nm and lower, and the other detector only registers wavelengths of 1100 nm and higher in wavelength. First, the speed of sequential pulsing into a single detector is limited by the time for each pulse to achieve a suitable peak followed by a return of the detector to a near quiet state or essentially its noise floor. Second, with the ability to increase the frequency of the pulse rate of the pulse pairs, the system can measure samples that exhibit a fast change nature such as high-speed flow in a process pipe or batch reactor. Third, and possibly most importantly, a higher degree of noise cancellation may be achieved with the simultaneous pulse pairs “seeing” the exact same particle environment such that the Raleigh, Mie, and geometric scattering of each wavelength pulse energy will match to a higher degree. For purposes of the present invention, near simultaneous pulsing will have near unity but recognizes that efficiency of pulsing may mean some difference in pulsing due to efficiency and inherent limits, and anything within approximately 5% to 10% will be deemed near simultaneous pulsing.

While the invention has been described herein with reference to certain preferred embodiments, those embodiments have been presented by way of example only, and not to limit the scope of the invention. Additional embodiments and further modifications are also possible in alternative embodiments that will be obvious to those skilled in the art having the benefit of this detailed description.

Accordingly, still further changes and modifications in the actual concepts described herein can readily be made without departing from the spirit and scope of the disclosed inventions.

What is claimed is:

1. A process for quantifying a concentration of a targeted molecule M in a liquid sample matrix through use of a sensor system comprised of electronic components, optical elements and software modules, comprising the steps of:
detecting, by at least one detector, a pulsed source signal, a pulsed interference signal, and a pulsed reference signal after they penetrate into a sample volume of the liquid sample matrix; and
obtaining, using signal processing,
i) a first average ratio value for a first preselected period of time from the pulsed source signal and the pulsed reference signal emerging from the sample volume and
ii) a second average ratio value for a second preselected period of time from the pulsed interference signal and pulsed reference signal; and
calculating, using electronics, the concentration of the targeted molecule M in the liquid sample matrix based on the first average ratio value for the first preselected period of time and a calibration curve validated using the second average ratio value for the second preselected period of time,
wherein a signal source emits radiation at a signal wavelength which is within a first absorption band of the targeted molecule M, an interference source emits radiation at an interference wavelength which is within a second absorption band of said at least one interfering molecule MJ, and a reference beam emits radiation at a reference wavelength which is neutral and is not within either the first absorption band or the second absorption band; and
wherein the pulsed source signal is circularly polarized.

2. The process of claim 1, wherein the pulsed reference signal is circularly polarized.

3. The process of claim 2, wherein the pulsed interference signal is circularly polarized.

4. The process of claim 1, wherein the pulsed source signal is configured to match a particular dichroism of the targeted molecule.

5. The process of claim 4, wherein the targeted molecule is the D-glucose enantiomer.

6. The process of claim 3, wherein the pulsed source signal is configured to match a particular dichroism of the targeted molecule.

7. The process of claim 6, wherein the targeted molecule is the D-glucose enantiomer.

8. The process of claim 3, wherein the pulsed interference signal is configured to match a particular dichroism selected for at least one interference targeted molecule in the liquid sample matrix.

9. The process of claim 1, wherein the pulsed source signal is produced by a quantum dot laser.

10. The process of claim 9 wherein the pulsed source signal is produced through use of polarization optics.

11. A system for quantifying a concentration of a targeted molecule M in a liquid sample matrix through use of a sensor system comprised of electronic components, optical elements and software modules, comprising:
a coherent laser signal source;
means for circularly polarizing a reference signal from the coherent laser signal source;
a coherent laser reference source;
a coherent laser interference source;
means for configuring pulsed radiation from the coherent laser source, the coherent laser reference source and the coherent laser interference source into a pulsed beam;
at least one detector configured to detect a pulsed source signal, a pulsed interference signal, and a pulsed reference signal after they penetrate into a sample volume of the liquid sample matrix;
signal processing for obtaining:
i) a first average ratio value for a first preselected period of time from the pulsed source signal and the pulsed reference signal emerging from the sample volume and
ii) a second average ratio value for a second preselected period of time from the pulsed interference signal and pulsed reference signal; and
electronics for calculating the concentration of the targeted molecule M in the liquid sample matrix based on the first average ratio value for the first preselected period of time and a calibration curve validated using the second average ratio value for the second preselected period of time,
wherein the coherent signal source emits radiation at a signal wavelength which is within a first absorption band of the targeted molecule M, the coherent interference source emits radiation at an interference wavelength which is within a second absorption band of said at least one interfering molecule MJ, and the coherent reference beam emits radiation at a reference wavelength which is neutral and is not within either the first absorption band or the second absorption band.

12. The system of claim 11, wherein the coherent laser signal source, the coherent reference source, the coherent interference source and the means for circularly polarizing a reference signal from the coherent laser signal source are implemented on a planar waveguide.

13. The system of claim 11, wherein the coherent laser signal source, the coherent reference source, the coherent interference source and the means for circularly polarizing a reference signal from the coherent laser signal source are implemented within a planar waveguide.

14. A system for quantifying a concentration of a targeted molecule M in a liquid sample matrix through use of a sensor system comprised of electronic components, optical elements and software modules, comprising:
an electronics front end comprising at least one signal generator, at least one laser driver, at least one TE cooler controller and at least one temperature sensor configured with a laser signal source, a laser reference source, a laser interference source, a combiner system and means for circularly polarizing at least a reference signal from the laser signal source; and
an electronics back end comprising:
(1) at least one detector configured to detect a pulsed source signal, a pulsed interference signal, and a pulsed reference signal after they penetrate into a sample volume of the liquid sample matrix;
(2) signal processing for obtaining i) a first average ratio value for a first preselected period of time from the pulsed source signal and the pulsed reference signal emerging from the sample volume and ii) a second average ratio value for a second preselected period of time from the pulsed interference signal and pulsed reference signal; and
(3) electronics for calculating the concentration of the targeted molecule M in the liquid sample matrix based on the first average ratio value for the first preselected period of time and a calibration curve validated using the second average ratio value for the second preselected period of time,
wherein the laser signal source emits radiation at a signal wavelength which is within a first absorption band of the targeted molecule M, the laser interference source emits radiation at an interference wavelength which is within a second absorption band of said at least one interfering molecule MJ, and the laser reference beam emits radiation at a reference wavelength which is neutral and is not within either the first absorption band or the second absorption band.

15. The system of claim 14 wherein the laser signal source, the reference source, the interference source and the means for circularly polarizing a reference signal from the coherent laser signal source are implemented on a planar waveguide.

16. The system of claim 14, wherein the laser signal source, the reference source, the interference source and the means for circularly polarizing a reference signal from the laser signal source are implemented within a planar waveguide.

17. The system of claim 14 wherein the system is contained in a single unitary package configured for allowing the concentration of the targeted molecule M to be quantified in the liquid sample matrix of a human body while the package is attached to or compressed against the human body.
